(19) United States
(12) Patent Application Publication
(10) Pub. No.: US 2022/0091116 A1
(43) Pub. Date: Mar. 24, 2022

(54) OPTICAL SENSING BASED ON FUNCTIONALIZED EVANESCENT FIBER SENSOR FOR PROCESS FLUID FLOW ANALYSIS

(71) Applicant: Ocean Optics, Inc., Orlando, FL (US)

(72) Inventor: Derek Guenther, St. Petersburg, FL (US)

(21) Appl. No.: 17/541,179

(22) Filed: Dec. 2, 2021

Related U.S. Application Data
(63) Continuation of application No. PCT/US20/36004, filed on Jun. 3, 2020.
(60) Provisional application No. 62/856,558, filed on Jun. 3, 2019.

(51) Int. Cl. G01N 33/543; G01N 21/77; G01N 21/64; G02B 6/036

(52) U.S. Cl. CPC G01N 33/54373; G02B 6/03622; G01N 21/648; G01N 21/7703

(57) ABSTRACT

Disclosed is an optical sensor device for detecting a chemical analyte including a light source configured to generate probe light having a first wavelength spectrum, an optical fiber sensor probe including a mechanically processed optical fiber segment which is chemically functionalized to include a sensing material formed on exterior of the fiber segment, the optical fiber sensor probe coupled to receive and guide the generated probe light inside the optical fiber sensor probe while allowing optical evanescent coupling between probe light guided inside the optical fiber sensor probe and the sensing material, and a detector coupled to the optical fiber sensor probe to optically detect the guided probe light to obtain information on a material property of the sensing material.

### CROSS-REFERENCE TO RELATED APPLICATIONS

This patent document is a continuation of International Application No. PCT/US20/36004, entitled “OPTICAL SENSING BASED ON FUNCTIONALIZED EVANESCENT FIBER SENSOR FOR PROCESS FLUID FLOW ANALYSIS” filed on Jun. 3, 2020 and which claims priority to and the benefits of U.S. Provisional Patent Application No. 62/856,558 entitled “OPTICAL SENSING BASED ON FUNCTIONALIZED EVANESCENT FIBER SENSOR FOR PROCESS FLUID FLOW ANALYSIS” filed on Jun. 3, 2019. The entire contents of the aforementioned patent application are incorporated by reference as part of the disclosure of this patent document.

### TECHNICAL FIELD

This patent document relates to optical sensing technologies.

### BACKGROUND

Optical sensors can use optical fiber either as the sensing element or as a means of relaying signals from a remote sensor to the electronics that process the signals. They allow direct measurements of liquids, powders, and flames, as well as solids.

### SUMMARY

Disclosed are methods, devices and applications pertaining to an optical fiber sensor having a mechanically processed and chemically functionalized optical fiber tip or optical fiber section to optically detect certain properties in a fluid flow based on optical evanescent coupling in transmissive or reflective mode.

In an embodiment of the disclosed technology, an optical sensor device for detecting a chemical analyte include a light source configured to generate probe light having a first wavelength spectrum, an optical fiber sensor probe including a mechanically processed optical fiber segment which is chemically functionalized to include a sensing material formed on exterior of the fiber segment, the optical fiber sensor probe coupled to receive and guide the generated probe light inside the optical fiber sensor probe while allowing optical evanescent coupling between probe light guided inside the optical fiber sensor probe and the sensed material, and a detector coupled to the optical fiber sensor probe to optically detect the guided probe light to obtain information on a material property of the sensed material.

In another embodiment of the disclosed technology, an optical fiber sensor for detecting a chemical analyte includes a first optical fiber segment including a first core and a first cladding surrounding the first core configured to cause light to be confined to the first core, a second optical fiber segment including: a second core connected to the first core; a second cladding surrounding the second core to cause an evanescent field to be generated at a boundary between the second core and the second cladding; and a sensing material layer disposed on the second cladding to cause the evanescent field to interact with the chemical analyte through the sensing material layer. The second cladding is thinner than the first cladding.

In another embodiment of the disclosed technology, a flow cell for process fluid flow analysis includes a liquid flow path through which an analyte flows, an optical path through which a waveguide is arranged to direct a light beam toward the analyte, and an evanescent fiber segment arranged at a crosspoint between the liquid flow path and the optical path to optically detect properties of the analyte. The evanescent fiber segment includes a fiber having a partially removed cladding on a core and a sensing material layer disposed on the partially removed cladding.

Those and other implementations and features of the disclosed technology are described in more detail in the drawings, the description and the claims.

### BRIEF DESCRIPTION OF THE DRAWINGS

FIG. 1A shows an example optical fiber sensor for detecting a chemical analyte implemented based on some embodiments of the disclosed technology.

FIG. 1B shows an example optical fiber sensor having one “knurled” or “roughened” fiber segment and another example optical fiber sensor having more than one “knurled” or “roughened” fiber segments.

FIG. 1C shows a probe of the optical fiber sensor that includes a roughened segment with pH responsive optical coating and a reflective silver paint layer at one end of the optical fiber.

FIG. 1D shows a sectional view of an example optical fiber sensor.

FIG. 2 shows an example configuration of an optical sensor device for detecting a chemical analyte.

FIG. 3A shows an example configuration of a flow cell for process fluid flow analysis.

FIG. 3B shows an example of the flow cell implemented using two subminiature assembly (SMA) units.

FIG. 3C shows an example of a reflective end of a fiber segment implemented using silver paint and room-temperature-vulcanizing (RTV) silicone.

FIG. 4 shows an absorbance response of bromocresol green dye across pH buffers where pH 1 is used as light reference.

FIG. 5A shows a maximum absorbance comparison of evanescent pH probes with a single knurled segment of variable length.

FIG. 5B shows an absorbance comparison of evanescent pH probes with a single knurled segment of variable length.

FIG. 5C shows an absorbance response of 1 mm knurled evanescent pH probes with various numbers of segments.

### DETAILED DESCRIPTION

There is a need to more reliably and affordably monitor pH in biopharmaceutical flow processes, and spectral interrogation using low-cost sensory films is a way to achieve this. However, some existing optical methods direct probe light to pass through a fluid flow to be measured. This direct interaction between the probe light and the fluid can lead to changes in the probe light by changes associated with the pH level of the fluid and also changes by other fluid properties not associated with the pH level, such as influence from sample color, turbidity, cross-fluorescence, and others. This direct interaction between the probe light and the fluid creates the potential for optical errors in pH measurement from sample color, turbidity and sediment, cross-fluorescence, etc. Some corrective techniques can be applied to account for these variations and provide the appropriate corrections but various corrective measures may not be able to fully remove such non-pH influences.

The technology disclosed in this patent document provides an optical waveguide such as a fiber probe to spatially confine the probe light inside the fiber probe by inserting the fiber probe into a target fluid to be measured without directing the probe light into the target fluid. A section of the exterior surface of the fiber probe is processed to have a pH-sensitive material which is in direct contact with the target fluid and will change an optical property of the material in response to a change in the pH value of the fluid. This change in the optical property of the material, when located in the evanescent field reach of guided probe light in the fiber probe, can be optically detected by and carried by the guided probe light if the fiber probe is structured to permit such evanescent interaction. The guided probe light in the fiber probe, upon evanescently interacting with the pH-sensitive material, carries information on the change in the pH level of the fluid imparted to the change in the optical property of the material and remains in the fiber probe without being in contact with the fluid. Optical detection of the guide probe light in the fiber probe can be performed to measure the pH level of the fluid.

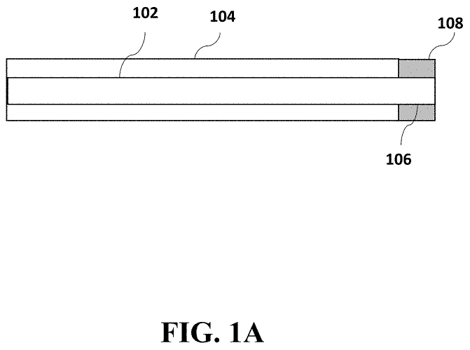

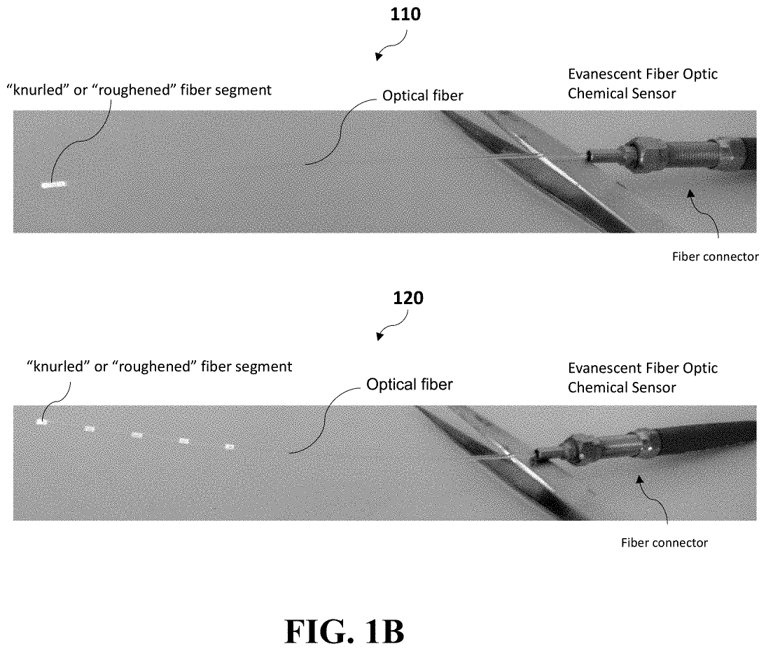

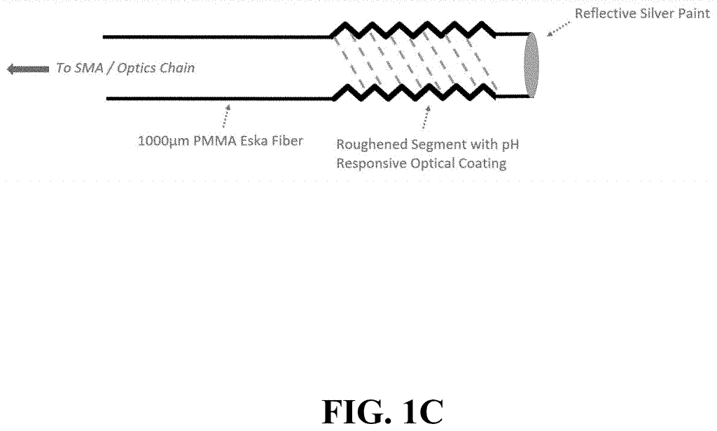

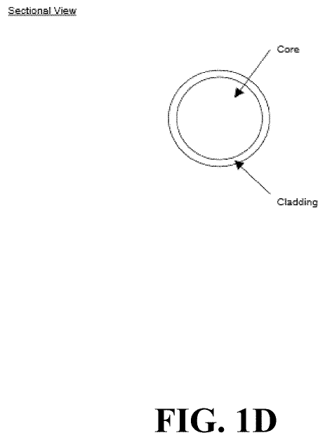

FIG. 1A shows an example optical fiber sensor for detecting a chemical analyte implemented based on some embodiments of the disclosed technology. FIG. 1B shows an example optical fiber sensor having one “knurled” or “roughened” fiber segment and another example optical fiber sensor having more than one “knurled” or “roughened” fiber segments. FIG. 1C shows an example configuration of an optical sensor device for detecting a chemical analyte. FIG. 1D shows a sectional view of an example optical fiber sensor.

As shown in FIG. 1A, a probe of the optical fiber sensor can be implemented using a strand of optical fiber with one or more segments that are mechanically processed and chemically functionalized. For example, a cladding 104 surrounding a core 102 of an optical fiber can be partially or entirely removed to form a “knurled” or “roughened” fiber segment 106 on which an optically responsive coating 108 is to be formed. Here, the “knurled” or “roughened” fiber segment 106 can be formed by at least partially removing a part of the cladding 104. The optically responsive coating may include any materials that can optically respond to chemical analytes. The roughening achieves increased interaction between the light and optical coating, as shown in FIG. 1B with 1-segment 110 and 5-segment assemblies 120. FIG. 1C shows a probe of the optical fiber sensor that includes a roughened segment with pH responsive optical coating and a reflective silver paint layer at one end of the optical fiber. As shown in FIG. 1D,the optical fiber sensor implemented based on some embodiments of the disclosed technology includes a core of an optical fiber and a cladding surrounding the core, and one or more portions of the cladding surrounding the core of the optical fiber can be removed to form a roughened fiber segment on which an optically responsive coating is to be formed.

An example specification of the optical fiber sensor is shown in Table 1 below:

TABLE 1

| | | | CK-40 | | |
|---|---|---|---|---|---|
| | | Specification | | | |
| Item | | Unit | Min. | Typ. | Max. |
| Optical | Core Material | — | Polymethyl-Methacrylate Resin | | |
| Fiber | Cladding Material | — | Fluorinated Polymer | | |
| | Core Refractive Index | — | 1.49 | | |
| | Refractive Index | — | Step Index | | |
| | Profile | | | | |
| | Numerical Aperture | — | 0.5 | | |
| | Core Diameter | μm | 920 | 980 | 1,040 |
| | Cladding Diameter | μm | 940 | 1,000 | 1,080 |

| | | |
|---|---|---|
| Approximate Weight | g/m | 1 |

The performances of the optical fiber sensor implemented based on the example specification above are shown in Table 2 below:

TABLE 2

| | | Acceptance Criterion | CK-40 | | | |
|---|---|---|---|---|---|---|
| | | and/or | Specification | | | |
| Item | | [Test Condition] | Unit | Min. | Typ. | Max. |
| Maximum | Storage | No Physical Deterioration | ° C. | −55 | — | +70 |
| Rating | Temperature | [in a Dry atmosphere] | | | | |
| | Operation | No Deterioration | ° C. | −55 | — | +70 |
| | Temperature | in Optical Properties* | | | | |
| | | [in a Dry atmosphere] | | | | |
| | | No Deterioration | ° C. | — | — | +80 |
| | | in Optical Properties** | | | | |
| | | [under 95% RH condition] | | | | |
| Optical | Transmission Loss | [650 nm Collimated Light] | dB/km | — | — | 200 |
| Properties | | [Standard condition] | | | | |
| | | [10 m-1 m cutback] | | | | |
| Mechanical | Minimum | Loss Increment ≤0.5 dB | mm | 25 | — | — |
| Characteristics | Bend Radius | [A Quarter Bend] | | | | |
| | Tensile Strength | Tensile Force at yield point | N | 65 | — | — |
| | | [in Conformity | | | | |
| | | to the JIS C 6861] | | | | |

All tests are carried out under temperature of 25° C. unless otherwise specified.
*Attenuation change shall be within +/−10% after 1,000 hours.
**Attenuation change shall be within +/−10% after 1,000 hours, except that due to absorbed water.

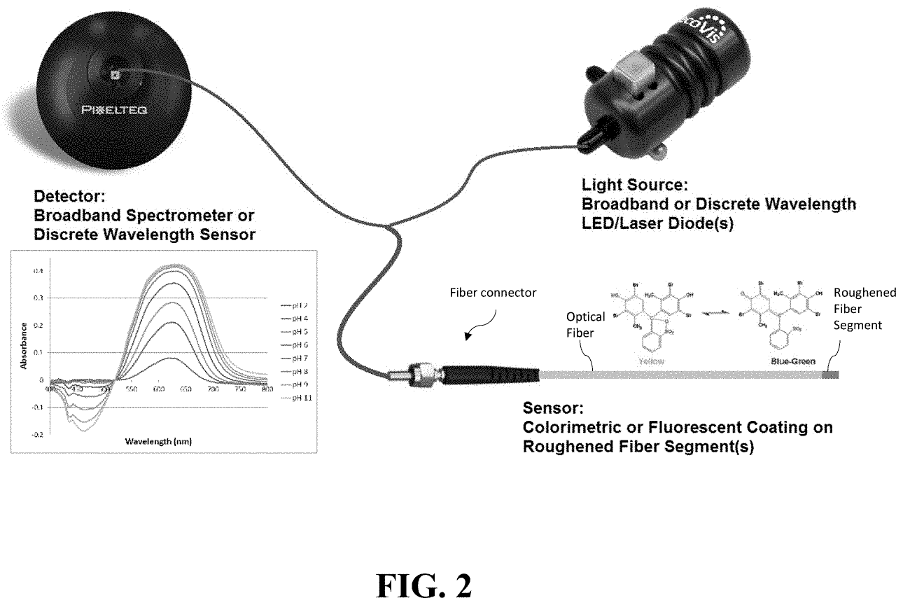

FIG. 2 shows a basic setup of the operation of the optical fiber sensor implemented based on some embodiments of the disclosed technology. The basic setup of the operation of the optical fiber sensor may include a light source, a detector, and a sensor. In an embodiment of the disclosed technology, the sensor includes an optical fiber that includes one or more roughened fiber segments and a fiber connector structured to connect the optical fiber to the light source and the detector. In some implementations, the fiber may be made as a loop (not shown) coupled with input/output ends of the fiber connector. In some implementations, a light beam is fed into a strand of optical fiber and is directed to the sensor with one or more roughened fiber segments with colorimetric or fluorescent coating formed thereon.

The optical fiber sensor implemented based on some embodiments of the disclosed technology can bypass interferences and provide a more stable measurement of the fluid by isolating photons within the optical fiber. The analytical light no longer directly enters the process fluid or uses it as a medium. Rather, the bulk of the photons reside within the installed sensory fiber (e.g., one or more roughened fiber segments with colorimetric or fluorescent coating), and the evanescent interaction with the color-changing sensor film is captured and relayed back to the detector. This provides a more stable measurement which will not falter as optical properties of the process fluid change.

The optical fiber sensor implemented based on some embodiments of the disclosed technology can be used for pH sensor chemistry (colorimetric, bromocresol green), moisture sensor chemistry (colorimetric, cobalt chloride degrees of hydration), and oxygen sensor chemistry (fluorescent, ruthenium and platinum porphyrins).

In an embodiment of the disclosed technology, the functionalized optical evanescent sensor can be implemented to deal with transmissive measurements. In another embodiment of the disclosed technology the functionalized optical evanescent sensor can be implemented to deal with reflective measurements by using a reflective layer of the optical fiber segment. Reflective material added to tip can make probe extremely immune to movement, ambient light, and sample color and turbidity. Unlike typical evanescent, side-coated fibers/sensors, low-cost plastic fibers can be used to achieve strong absorbance signals through just several fiber treatments. In some implementations, a roughened portion of a plastic fiber can be used to achieve an evanescent absorbance measurement.

The optical sensor implemented based on some embodiments of the disclosed technology includes a mechanically processed and chemically functionalized optical fiber segment to utilize evanescent waves in measuring characteristics of chemical analytes. This measurement is based on the interaction between the evanescent wave and the surrounding environment. When light passes through the mechanically processed (e.g., side-polished) optical fiber segment, a fraction of the radiation can extend a small distance (an evanescent field) from the mechanically processed region. This evanescent wave can interact with the chemical analytes through a chemically functionalized layer disposed on a mechanically processed side of the optical fiber. The evanescent field that enters a waveguide from the mechanically processed and chemically functionalized optical fiber segment can be collected by a detector to analyze the characteristics of the chemical analytes.

Some embodiments of the disclosed technology can be implemented to utilize a mechanically processed and chemically functionalized optical fiber installed into a liquid flow cell for interrogation of some aspect of the fluid. A fiber made of plastic or glass is roughened or “knurled” around the outer circumference of the fiber for a specified segment length, which may vary and can also include multiple segments. In an example, a 1000 μm plastic (PMMA) fiber can be used and it can be roughened using 280-grit barrel sanders.

This roughened portion of the fiber is functionalized or coated with an optically active sensory film. Some embodiments of the disclosed technology can be used to implement an optical pH sensor using colorimetric pH sol-gel formulation, moisture/humidity-sensitive colorimetric compounds, and/or oxygen-sensitive fluorescent compounds. The optical pH sensor is coupled to a light source. Light generated at the light source is transmitted to a mechanically processed and chemically functionalized optical fiber segment of the optical pH sensor. Based on the pH of the chemical analytes, a certain amount of light may absorb at a certain wavelength range or ranges. Such partially absorbed light travels to the detector and is compared with a previously taken reference to obtain a pH value based on a predetermined algorithm. For example, in the case where the indicator molecules absorb light when exposed to a basic solution, the partially absorbed light is compared to a reference taken as a zero absorbance across the entire spectrum to represent all indicator molecules in an acid form.

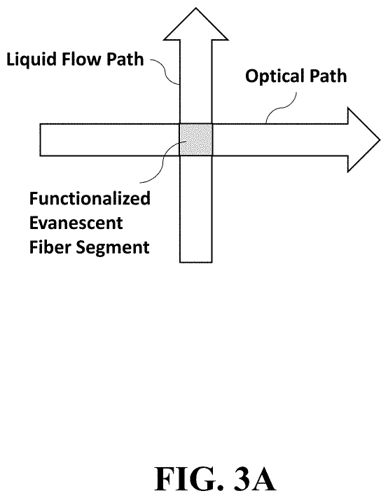

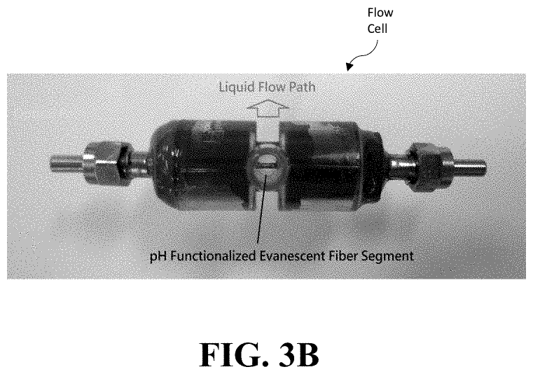

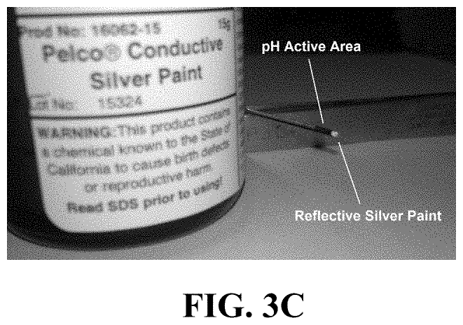

FIG. 3A shows an example configuration of a flow cell for process fluid flow analysis. FIG. 3B shows an example of the flow cell implemented using two subminiature assembly (SMA) units. FIG. 3C shows an example of a reflective end of a fiber segment implemented using silver paint and room-temperature-vulcanizing (RTV) silicone.

After the sensor film has cured on the roughened portion, the fiber may be mechanically integrated into a flow cell form factor in transmissive or reflective mode. The roughened sensory portion is positioned such that it is in contact with the process fluid. An example schematic of the flow cell is shown in FIG. 3A. The flow cell implemented based on some embodiments of the disclosed technology may include at least one optical coupling. As shown in FIG. 3B, the flow cell implemented using two subminiature assembly (SMA) units may include two points of optical coupling. In some embodiments of the disclosed technology, the fiber segment implemented in the flow cell can include a reflective end within the flow cell for optical signals to be directed to a detector. In an implementation, the reflective end of the fiber segment can be formed by terminating the fiber within the flow cell with an optically reflective layer as well as a sealing layer. As shown in FIG. 3C, the reflective end of the fiber segment can be formed using silver paint and room-temperature-vulcanizing (RTV) silicone. The optical fiber segment with a reflective termination may reduce the number of optical interfaces from 2 to 1, and may provide more reliable absorbance measurements of the sensor chemistry. Some embodiments of the disclosed technology can create a very slight recess, which allows tight fitment when installed into a liquid port such as in a flow cell.

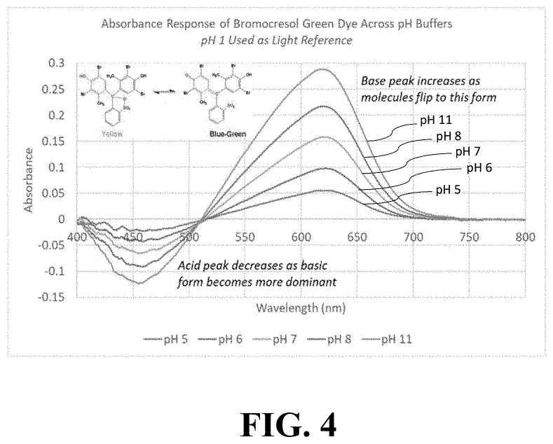

FIG. 4 shows an absorbance response of bromocresol green dye across pH buffers where pH 1 is used as light reference. When fiber-coupled to a light source and spectrometer system, the light will interact with the pH-sensitive coating along the roughened segment(s) and relay this optical information to the detector. The pH sensors built for these prototypes shift from yellow in the acidic range to blue in the basic range, with large optical shift occurring around 620 nm.

Pharmaceutical processes are notorious for requiring precise monitoring and control of pH to ensure desired products and yields are obtained. The mechanically processed and chemically functionalized optical fiber tip or fiber section implemented based on some embodiments of the disclosed technology can be used in pharmaceutical processes to offer a fast and consumable approach to pH monitoring in such a process environment.

In some embodiments of the disclosed technology, the functionalized optical evanescent sensor can be used for real-time monitoring of process fluids in their flow condition. The immediate use described here pertains to optical sensing of fluid pH. This provides a low-cost and minimal-interface approach to optical monitoring of a process fluid flow. The components can be integrated into disposable plastic flow cells, and can be easily coupled to more permanent detection hardware. The evanescent interrogation of the sensory coating avoids many of the downfalls seen with traditional transmissive approaches, including noise/errors from sample turbidity, color, and other interferences.

In addition to the pH sensor prototypes discussed here, functioning sensors implemented based on some embodiments of the disclosed technology may also be built for the optical detection of molecular oxygen and of humidity/moisture/aqueous-content. This may quickly be expanded into other desired analytes.

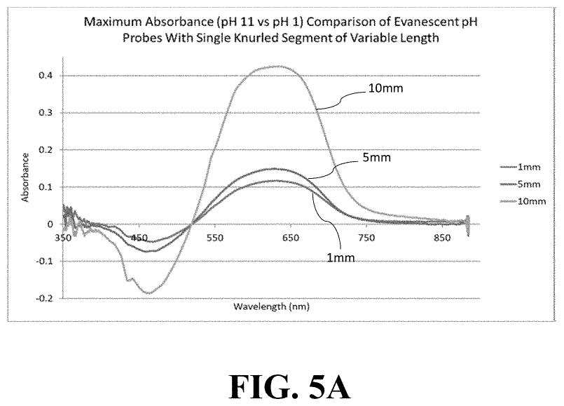

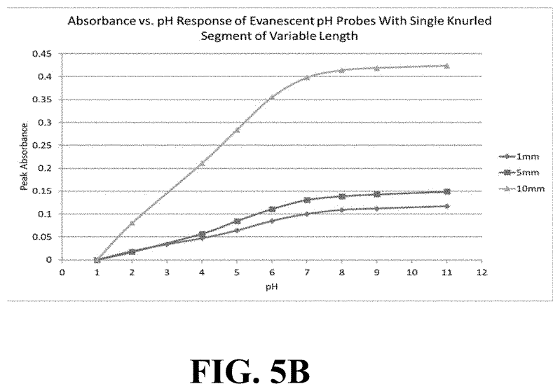

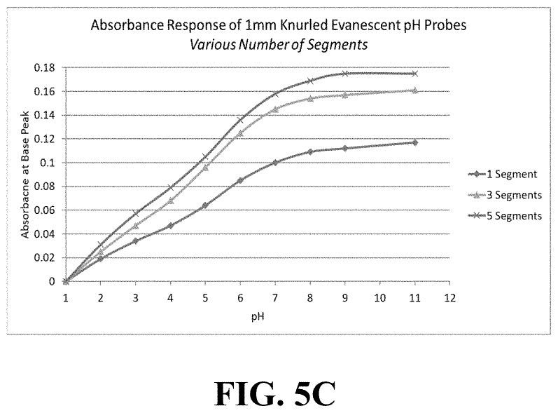

FIG. 5A shows a maximum absorbance comparison of evanescent pH probes with a single knurled segment of variable length. FIG. 5B shows an absorbance comparison of evanescent pH probes with a single knurled segment of variable length. FIG. 5C shows an absorbance response of 1 mm knurled evanescent pH probes with various numbers of segments.

In FIGS. 5A-5C, the plots show the absorbance response of the approach using various lengths of roughening and pH sensor coating (1 mm, 5 mm, 10 mm). The first plot shows the broadband response of the active regions, and the second plot shows the base peak absorbance response across integer pH buffers. Furthermore, the ability to use multiple segments at different locations along the fiber was investigated, and also showed success and an expected trend.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed are techniques and structures as described and shown, including:

1. An optical sensor device for detecting a chemical analyte, comprising:
a light source configured to generate probe light having a first wavelength spectrum;
an optical fiber sensor probe including a mechanically processed optical fiber segment which is chemically functionalized to include a sensing material formed on exterior of the fiber segment, the optical fiber sensor probe coupled to receive and guide the generated probe light inside the optical fiber sensor probe while allowing optical evanescent coupling between probe light guided inside the optical fiber sensor probe and the sensing material; and
a detector coupled to the optical fiber sensor probe to optically detect the guided probe light to obtain information on a material property of the sensing material.

2. The device of claim 1, wherein the optical fiber segment includes a fiber segment having a partially removed cladding on a core, wherein the sensing material is disposed on the partially removed cladding.

3. The device of claim 2, wherein the sensing material includes a colorimetric pH sol-gel formulation, a moisture/humidity-sensitive colorimetric compound, or an oxygen-sensitive fluorescent compound, or a combination of any two or more of the colorimetric pH sol-gel formulation, the moisture/humidity-sensitive colorimetric compound, and the oxygen-sensitive fluorescent compound.

4. The device of claim 1, wherein the optical fiber sensor further comprises a reflective layer at a termination thereof

5. The device of claim 4, wherein the reflective layer includes a reflective metal material.

6. The device of claim 1, wherein the sensing material reacts to a change in a pH level of a fluid to allow for measurement of the pH level of the fluid by detecting the guided probe light after evanescently interacting with the sensing material which is in contact with the fluid without having the probe light to be in contact with the fluid.

7. The device of claim 1, wherein the optical fiber sensor probe further includes a fiber connector and an optical fiber, the fiber connector structured to couple the optical fiber to the light source and the detector, the optical fiber structured to connect the optical fiber segment to the fiber connector.

8. The device of claim 7, wherein the optical fiber is structured to include a loop connected to input and output ends of the fiber connector.

9. An optical fiber sensor for detecting a chemical analyte, comprising:
a first optical fiber segment including a first core and a first cladding surrounding the first core configured to cause light to be confined to the first core; and
a second optical fiber segment including:
a second core connected to the first core;
a second cladding surrounding the second core to cause an evanescent field to be generated at a boundary between the second core and the second cladding; and
a sensing material layer disposed on the second cladding to cause the evanescent field to interact with the chemical analyte through the sensing material layer,
wherein the second cladding is thinner than the first cladding.

10. The sensor of claim 9, wherein the second cladding includes a mechanically processed surface structured to support the sensing material layer.

11. The sensor of claim 9, further comprising a reflective layer at a termination of second optical fiber segment.

12. The sensor of claim 9, wherein the sensing material layer includes a colorimetric pH sol-gel formulation, a moisture/humidity-sensitive colorimetric compound, or an oxygen-sensitive fluorescent compound, or a combination of any two or more of the colorimetric pH sol-gel formulation, the moisture/humidity-sensitive colorimetric compound, and the oxygen-sensitive fluorescent compound.

13. The sensor of claim 9, wherein the first and second optical fiber segments are coupled to receive and guide probe light inside the first and second cores while allowing optical evanescent coupling between the probe light guided inside the second core and the sensing material layer.

14. The sensor of claim 13, wherein the sensing material layer reacts to a change in a pH level of a fluid to allow for measurement of the pH level of the fluid by detecting the guided probe light after evanescently interacting with the sensing material layer which is in contact with the fluid without having the probe light to be in contact with the fluid.

15. A flow cell for process fluid flow analysis, comprising:
a liquid flow path through which an analyte flows;
an optical path through which a waveguide is arranged to direct a light beam toward the analyte; and
an evanescent fiber segment arranged at a cross point between the liquid flow path and the optical path to optically detect properties of the analyte, wherein the evanescent fiber segment includes a fiber having a partially removed cladding on a core and a sensing material layer disposed on the partially removed cladding.

16. The flow cell of claim 15, wherein the sensing material layer includes a colorimetric pH sol-gel formulation, a moisture/humidity-sensitive colorimetric compound, or an oxygen-sensitive fluorescent compound, or a combination of any two or more of the colorimetric pH sol-gel formulation, the moisture/humidity-sensitive colorimetric compound, and the oxygen-sensitive fluorescent compound.

17. The flow cell of claim 15, wherein the core of the fiber coupled to guide the light beam inside the core while allowing optical evanescent coupling between the light beam guided inside the core and the sensing material layer.

18. The flow cell of claim 17, wherein the sensing material layer reacts to a change in a pH level of the analyte to allow for measurement of the pH level of the analyte by detecting the guided light beam after evanescently interacting with the sensing material layer which is in contact with the analyte without having the light beam to be in contact with the analyte.

19. The flow cell of claim 15, wherein the core from which the cladding is partially removed includes a mechanically processed surface structured to support the sensing material layer.

20. The flow cell of claim 15, wherein the evanescent fiber segment further comprises a reflective layer at one end thereof.
